(19) United States
(12) Patent Application Publication
(10) Pub. No.: US 2013/0190624 A1
(43) Pub. Date: Jul. 25, 2013

(54) METHOD AND APPARATUS FOR DISPLAYING AN ULTRASOUND IMAGE

(71) Applicant: Aesculap AG, Tuttlingen (DE)

(72) Inventors: Jens Beger, Tuttlingen (DE); Josef Kozak, Tuttlingen (DE)

(73) Assignee: AESCULAP AG, Tuttlingen (DE)

(21) Appl. No.: 13/745,937

(22) Filed: Jan. 21, 2013

(30) Foreign Application Priority Data
Jan. 23, 2012 (DE) 10 2012 100 504

(51) Int. Cl. A61B 8/00

(52) U.S. Cl. CPC A61B 8/461; A61B 8/42
USPC 600/443

(57) ABSTRACT

A method for displaying an ultrasound image on a display device is provided. Image signals are generated with an ultrasound probe during examination of an item under examination with ultrasound and are transferred to a data processing device. The image signals are displayed by the data processing device as an ultrasound image on a display device. In order to make it easier for an operator to observe the ultrasound image, by means of an orientation detection device, a change in orientation of the display device in space is detected and an associated change in orientation signal is provided to the data processing device and the orientation of the ultrasound image on the display device is changed by the data processing device as a function of the change in orientation of the display device in space. An apparatus for carrying out the method is also provided.

### CROSS-REFERENCE TO RELATED APPLICATIONS

This application claims the benefit of German application number DE 10 2012 100 504.4, filed Jan. 23, 2012, which is incorporated herein by reference in its entirety and for all purposes.

### FIELD OF THE INVENTION

The present invention relates to a method for displaying an ultrasound image on a display device, in which image signals are generated with an ultrasound probe during examination of an item under examination with ultrasound and are transferred to a data processing device and the image signals are displayed by the data processing device as an ultrasound image on a display device.

Moreover, the present invention relates to an apparatus for displaying an ultrasound image on a display device, in particular for carrying out the above-stated method, comprising an ultrasound probe for generating image signals during examination of an item under examination with ultrasound, a data processing device to which the image signals are transferable and on which the image signals are processable, and a display device which is suppliable by the data processing device with ultrasound image information for displaying an ultrasound image.

### BACKGROUND OF THE INVENTION

It is known to produce two-dimensional images of items under examination with ultrasound probes and to display said images as an ultrasound image on a display device. The ultrasound probe conventionally comprises a plurality of ultrasound transmitters arranged in a linear array which in each case emit ultrasound radiation and consequently generate an ultrasound image which conventionally defines a plane. The emitted ultrasound radiation penetrates into the item under examination, is reflected on specific structures thereof and received back by the ultrasound probe, the distance of the reflective structure from the ultrasound probe being determined by the echo time of the ultrasound radiation between emission thereof and reception by the ultrasound probe. The ultrasound image is generally a two-dimensional representation of the reflective structure. Starting from upper edge of the ultrasound image, which corresponds to an examination zone adjacent the linear array of ultrasound transmitters, the propagation time of the ultrasound beam and thus the distance of the reflective structures from the ultrasound transmitters is displayed downwardly. The transverse direction of the ultrasound image corresponds to the spatial arrangement of the reflective structures lengthwise or parallel to the linear arrangement of ultrasound transmitters.

The ultrasound image is typically displayed in the plane of the ultrasound probe in which the ultrasound radiation is emitted. For example, the left-hand side of the display device is associated with the left-hand side of the ultrasound probe and the right-hand side of the display device to the right-hand side of the ultrasound probe.

However, if the orientation of the ultrasound probe changes relative to the item under examination, for instance in the event of rotation, tilting or panning of the probe, this association remains unchanged. This may on occasion make it more difficult for an operator to establish how the ultrasound probe is oriented relative to the item under examination. In order to make this easier for an operator, DE 10 2006 024 629 B4 proposes determining the location of the ultrasound probe in space by means of a navigation system and supplying a corresponding signal to the data processing device which displays the ultrasound image on the display device. The data processing device keeps the orientation of the image constant if the change in orientation of the ultrasound probe in space is within a defined angular range but changes the orientation of the image on the display device if the ultrasound probe is moved out of this angular range into an adjacent angular range.

The method described in DE 10 2006 024 629 B4 does not take account of the fact that the operator may move relative to the display device and that the operator's viewing angle onto the display device may change. Despite the advantage which may be achieved by the method mentioned in the above-stated document, associating structures recognisable in the ultrasound image with structures of the item under examination may be made more difficult.

An object underlying of the present invention is to provide a method of the generic type and an apparatus of the generic type which make it easier for an operator to observe the ultrasound image.

### SUMMARY OF THE INVENTION

In an aspect of the invention, a method is provided for displaying an ultrasound image on a display device, in which image signals are generated with an ultrasound probe during examination of an item under examination with ultrasound and are transferred to a data processing device and the image signals are displayed by the data processing device as an ultrasound image on a display device. By means of an orientation detection device, a change in the orientation of the display device in space is detected and an associated change in orientation signal is provided to the data processing device and the orientation of the ultrasound image on the display device is changed by the data processing device as a function of the change in orientation of the display device in space.

In another aspect of the invention, an apparatus for displaying an ultrasound image on a display device, in particular for carrying out the above-stated method, comprises an ultrasound probe for generating image signals during examination of an item under examination with ultrasound, a data processing device to which the image signals are transferable and on which the image signals are processable, and a display device which is suppliable by the data processing device with ultrasound image information for displaying an ultrasound image. The apparatus comprises a first orientation detection device for detecting a change in orientation of the display device in space and transferring an associated change in orientation signal to the data processing device. The data processing device is configured and programmed such that it changes the orientation of the ultrasound image on the display device as a function of the change in orientation of the display device in space.

### BRIEF DESCRIPTION OF THE DRAWING FIGURES

The foregoing summary and the following description may be better understood in conjunction with the drawing figures, of which:

FIG. 1: is a schematic diagram of a preferred embodiment of an apparatus according to an aspect of the invention comprising an ultrasound probe, with which an item under examination in the form of a femur is examined, and of an integrated display and data processing device on which an ultrasound image is displayed;

FIG. 2: shows the apparatus from FIG. 1, the display device being illustrated rotated by a first angle of rotation;

FIG. 3: shows the apparatus from FIG. 1, the display device being illustrated rotated by a second angle of rotation;

FIG. 4: shows the apparatus from FIG. 1, the ultrasound probe and the ultrasound image in each case being illustrated rotated by an angle of rotation;

FIG. 5: shows the apparatus from FIG. 1, the ultrasound probe examining the same examination zone of the femur but being illustrated rotated by 180° and

FIG. 6: shows a further preferred embodiment of the apparatus according to an aspect of the invention.

### DETAILED DESCRIPTION OF PREFERRED EMBODIMENTS OF THE INVENTION

Although the invention is illustrated and described herein with reference to specific embodiments, the invention is not intended to be limited to the details shown. Rather, various modifications may be made in the details within the scope and range of equivalents of the claims and without departing from the invention.

The present invention relates to a method for displaying an ultrasound image on a display device, in which image signals are generated with an ultrasound probe during examination of an item under examination with ultrasound and are transferred to a data processing device and the image signals are displayed by the data processing device as an ultrasound image on a display device. By means of an orientation detection device, a change in the orientation of the display device in space is detected and an associated change in orientation signal is provided to the data processing device and the orientation of the ultrasound image on the display device is changed by the data processing device as a function of the change in orientation of the display device in space.

The method according to an aspect of the invention provides detecting when the orientation of the display device in space changes. A corresponding change in orientation signal may be supplied to the data processing device. The data processing device is capable of changing the ultrasound image on the display device as a function of the change in orientation. This for example makes it possible to use a display device which is designed to be movable in such a manner that it may be positioned as required by the operator such that the ultrasound image is readily visible. For example, the display device may be positioned such that the ultrasound image is displayed for the operator in the plane of the ultrasound probe. A change in orientation of the ultrasound image on the display device by the data processing device may in particular proceed in such a manner that, in the event of a change in orientation of the display device, the ultrasound image is maintained for the operator. For example, in this way a user-oriented display of the ultrasound image may be achieved which, in the event of a change in orientation of the display device in space, is stationary relative to the operator. This makes it considerably easier for an operator to recognise the ultrasound image and to associate structures contained therein with structures of the item under examination. A display device which is freely movable in space, for example included in a tablet computer or the like, is preferably used in the method according to an aspect of the invention. This enables the operator to position the display device flexibly in space in such a manner that he/she has the best possible view of the ultrasound image.

The ultrasound image may, for example, be rotated as a function of rotation of the display device in space.

It is favourable for the ultrasound image to be rotated in the opposite direction to the rotation of the display device, preferably by the same angular amount, about an axis of rotation oriented perpendicularly to ultrasound image plane. This makes it possible to display the ultrasound image on the display device stationarily relative to the operator.

Advantageously, the ultrasound image is not rotated if the angle of rotation of the display device is below a predeterminable threshold angle of rotation and is only rotated once the angle of rotation exceeds the threshold angle of rotation. In the event of an only slight rotation of the display device by an angle of rotation below the threshold angle of rotation, the image is kept constant on the display device. This is in particular advantageous in the event of an intrinsically unintentional rotation of the display device, for example due to its being knocked.

The orientation of the ultrasound image is preferably not changed if the display device is rotated about an axis of rotation which extends in the ultrasound image plane, i.e. if the display device is inclined or tilted relative to the original ultrasound image plane. In this way, a user-oriented display of the ultrasound image on the display device may for example be kept constant.

In the event of a movement of the display device in space such that it is moved parallel relative to the original ultrasound image plane, but no rotation occurs, it is advantageous for there to be no change in orientation of the ultrasound image.

If the display device is panned about an axis which is oriented obliquely relative to the ultrasound image plane, this may be attributed to a superimposed movement, namely rotation of the display device about an axis oriented perpendicularly to the ultrasound image plane, about at least one axis extending in the ultrasound image plane, optionally together with a parallel movement of the display device in space. A possible change in orientation of the ultrasound image on the display device may then be identified by considering the change in orientation of the ultrasound image as a function of the rotation about the axes perpendicular to or in the ultrasound image plane and as a function of the parallel movement of the display device.

It is favourable for the change in orientation of the ultrasound image to proceed as a function of a change in orientation of the display device subject to an actuating element being actuated by an operator, without which actuation the orientation of the ultrasound image is kept constant even in the event of a change in orientation of the display device in space. This enables the operator to set a display mode of the display device in such a manner that the ultrasound image is changed selectively. As a consequence, the method exhibits greater versatility. It may for example be provided for the operator initially to position the display device in a manner advantageous to the operator and then to actuate the actuating element, whereupon the orientation of the ultrasound image is changed as a function of the change in orientation of the display device.

An orientation detection device included in or arranged on the display device is preferably used to detect a change in orientation of the display device in space, the display device and the data processing device favourably being integrated in one another. “Integrated in one another” may in the present case in particular mean “located in a common device”. This proves advantageous for example when using a tablet computer or the like which includes the display device. The orientation detection device for example comprises an inclination sensor and/or a compass sensor for detecting a change in orientation of the display device.

It may alternatively or additionally be provided that an orientation detection device spatially separate from the display device be used in order to detect the change in orientation of the display device in space. It is here in particular possible for a marker device to be fixed to the display device or for the latter to comprise such a marker device, the change in orientation of which in space is detected by the orientation detection device. It may for example be provided for this purpose that the marker device emit and/or reflect electromagnetic radiation which is detected by the orientation detection device which may for this purpose comprise a camera.

The orientation of the ultrasound image on the display device is preferably selected such that the ultrasound image is oriented on the display device parallel to the ultrasound field emitted by the ultrasound probe, relative to a projection of the ultrasound image on a plane defined by the ultrasound field. This makes it possible to display the ultrasound image in the plane of the ultrasound field in such a manner that for example the left-hand and the right-hand side of the ultrasound image are respectively associated with the left-hand and right-hand side of the ultrasound field, and the top side and the bottom side of the ultrasound image are respectively associated with the beginning and end of the ultrasound field (smallest and greatest distances from the ultrasound transmitters and ultrasound receivers). It may thus be ensured that the ultrasound image is displayed in a fixed geometric relationship to the ultrasound field. This enables the operator more straightforwardly to associate structures of the ultrasound image with structures of the item under examination.

It is advantageous if, by means of an orientation detection device, a change in orientation of the ultrasound probe in space is detected and an associated change in orientation signal is transferred to the data processing device and if the orientation of the ultrasound image on the display device is changed as a function of the change in orientation of the ultrasound probe in space. This additionally makes it simpler for an operator to associate structures in the ultrasound image with structures of the item under examination. In this manner, it is in particular possible not only to enable a user-oriented display of the ultrasound image, but it is furthermore at the same time also possible to provide a device-oriented display of the ultrasound image, which display changes in the event of a change in orientation of the ultrasound probe in space.

For example, the ultrasound image is rotated on the display device as a function of rotation of the ultrasound probe in space.

In particular, it may advantageously be provided that, in the event of rotation of the ultrasound probe about an axis of rotation perpendicular to a plane defined by the ultrasound field, the ultrasound image on the display device be rotated about an axis of rotation oriented perpendicularly to the ultrasound image plane, in the same direction of rotation and preferably by the same angular amount. For example, if the ultrasound probe is rotated rightwards relative to an operator, the ultrasound image on the display device is likewise rotated rightwards and vice versa, the geometric relationship of the ultrasound image to the geometry of the ultrasound field preferably being maintained.

It may furthermore be provided that the image not be rotated if the angle of rotation of the ultrasound probe is below a predeterminable threshold angle of rotation and only be rotated once the angle of rotation exceeds the threshold angle of rotation, such that, in the event of slight rotational movements of the ultrasound probe which fall below the threshold angle of rotation, there is no change to the ultrasound image. This makes it easier for an operator to recognise the ultrasound image.

In the event of rotation of the ultrasound probe by 180° or substantially 180° about an axis of rotation which extends centrally in the plane of the ultrasound field, it is advantageous for the ultrasound image on the display device to be mirrored about a mirror plane perpendicular to the ultrasound image plane, which is oriented parallel to or contains the axis of rotation and extends through the centre of the ultrasound image. This makes it possible to provide two left/right orientation options and/or two top/bottom orientation options for the ultrasound image. If, for example, the ultrasound probe is rotated by 180° about an axis of rotation oriented centrally in the ultrasound field along the direction of propagation of the ultrasound radiation and thus the left-hand side of the ultrasound probe is, as it were, swapped with the right-hand side, the ultrasound image observable by the operator remains unchanged. This saves the operator the task of mirroring the ultrasound image in his/her head, this instead being carried out by mirroring of the ultrasound image on the display device. A corresponding situation applies when the ultrasound probe is rotated by 180° about an axis of rotation which is oriented perpendicularly to direction of propagation the ultrasound radiation and extends in the plane of the ultrasound field.

An orientation detection device included in or arranged on the ultrasound probe is preferably used in order to detect the change in orientation of the ultrasound probe in space. The orientation detection device comprises for example an inclination sensor and/or a compass sensor for detecting the change in orientation. A corresponding change in orientation signal may be supplied to the data processing device, together with the image signals for the ultrasound image.

It may alternatively or additionally be provided that an orientation detection device spatially separate from the ultrasound probe be used in order to detect the change in orientation of the ultrasound probe in space. For example, a marker device arranged on or included in the ultrasound probe is used which is capable of emitting and/or reflecting electromagnetic radiation, the electromagnetic radiation being detectable by the orientation detection device and an associated change in orientation signal being transferrable to the data processing device.

Moreover, the present invention relates to an apparatus for displaying an ultrasound image on a display device comprising an ultrasound probe for generating image signals during examination of an item under examination with ultrasound, a data processing device to which the image signals are transferable and on which the image signals are processable, and a display device which is suppliable by the data processing device with ultrasound image information for displaying an ultrasound image. The apparatus comprises a first orientation detection device for detecting a change in orientation of the display device in space and transferring an associated change in orientation signal to the data processing device. The data processing device is configured and programmed such that it changes the orientation of the ultrasound image on the display device as a function of the change in orientation of the display device in space.

The method according to the aspect of the invention mentioned above may be carried out by means of the apparatus, and the advantages achievable by using the method according to the aspect of the invention mentioned above, to which reference is made in order to avoid repetition, may likewise be achieved by means of the apparatus.

The data processing device is for example configured and programmed such that it rotates the ultrasound image as a function of rotation of the display device in space.

It is favourable for the data processing device to be configured and programmed such that it rotates the ultrasound image on the display device in the opposite direction to the rotation of the display device, preferably by the same angular amount, about an axis of rotation oriented perpendicularly to the ultrasound image plane.

It may here in particular be provided that the data processing device be configured and programmed such that it does not rotate the ultrasound image if the angle of rotation of the display device is below a threshold angle of rotation and only rotates it once the angle of rotation exceeds the threshold angle of rotation.

The data processing device is advantageously configured and programmed such that it does not change the orientation of the ultrasound image on the display device if the display device is rotated about an axis of rotation which extends in the ultrasound image plane.

It is also favourable for the data processing device to be configured and programmed such that the orientation of the ultrasound image is not changed if the display device is moved parallel in space relative to the original ultrasound image plane, but no rotation occurs.

It is favourable for the apparatus to comprise an actuating element and for the data processing device to be configured and programmed such that it changes the orientation of the ultrasound image as a function of actuation of the actuating element, without which actuation the data processing device keeps the orientation of the ultrasound image constant even in the event of a change in orientation of the display device in space. The actuating element is arranged for example on the display device or on the data processing device.

The display device preferably comprises the first orientation detection device, the data processing device and the display device preferably being integrated in one another. This enables a spatially compact and simple design of the apparatus.

It may be provided that the first orientation detection device comprise at least one inclination sensor and/or at least one compass sensor for detecting the change in orientation of the display device in space.

It may alternatively or additionally be provided that the first orientation detection device be constructed spatially separately from the display device, that the display device comprise a marker device or such a marker device be arranged on the display device and that the orientation detection device preferably be configured for detecting radiation emitted and/or reflected from the marker device. This makes it possible for example to use a navigation system in which, by means of the orientation detection device, not only is a change in orientation of the display device in space detected, but also a change in orientation of further components of the apparatus in space, in particular of the ultrasound probe.

It is favourable for the data processing device to be configured and programmed such that it selects the orientation of the ultrasound image on the display device such that the ultrasound image is oriented parallel to the ultrasound field emitted by the ultrasound probe, relative to a projection of the ultrasound image on a plane defined by the ultrasound field. In this manner, as already mentioned, a fixed geometric relationship may be ensured between the ultrasound image and the ultrasound field, with for example the left-hand side of the ultrasound image being associated with the left-hand side of the ultrasound field and the right-hand side of the ultrasound image with the right-hand side of the ultrasound field.

It is advantageous for the apparatus to comprise a second orientation detection device for detecting a change in orientation of the ultrasound probe in space and transferring an associated change in orientation signal to the data processing device and for the data processing device to be configured and programmed such that it changes the orientation of the ultrasound image on the display device as a function of the change in orientation of the ultrasound probe in space. As already mentioned, in this way the ultrasound image may be displayed not only in user-oriented manner but additionally in device-oriented manner. This makes it easier for an operator to associate structures in the ultrasound image with structures of the item under examination.

It may be provided that the first and the second orientation detection device be integrated in one another. In particular, the first orientation detection device may entirely or in part include and/or form the second orientation detection device.

The data processing device is, for example, configured and programmed such that it rotates the ultrasound image on the display device as a function of rotation of the ultrasound probe in space.

Advantageously, the data processing device is configured and programmed such that, in the event of rotation of the ultrasound probe about an axis of rotation perpendicular to a plane defined by the ultrasound field, it rotates the ultrasound image on the display device about an axis of rotation oriented perpendicularly to the ultrasound image plane, in the same sense of rotation and preferably by the same angular amount.

It is favourable for the data processing device to be configured and programmed such that it does not rotate the image if the angle of rotation of the ultrasound probe is below a threshold angle of rotation and only rotates it once the angle of rotation exceeds the threshold angle of rotation, in order to keep the ultrasound image constant in the event of the ultrasound probe being turned by only a small angular amount and to make it easier for an operator to comprehend it.

It is advantageous for the data processing device to be configured and programmed such that, in the event of rotation of the ultrasound probe by 180° or substantially 180° about an axis of rotation which extends centrally in the plane of the ultrasound field and centrally therethrough, the ultrasound image on the display device is mirrored about a mirror plane perpendicular to the ultrasound image plane, which is oriented parallel to or contains the axis of rotation and extends through the centre of the ultrasound image. As already explained, it is consequently possible for there to be two left/right orientation options and/or two top/bottom orientation options for the ultrasound image relative to the geometry of the ultrasound probe. An operator is thereby saved the task of mirroring the ultrasound image mentally in order to associate structures of the ultrasound image with structures of the item under examination.

In order to achieve a compact and simple design of the apparatus, it may be provided that the ultrasound probe include the second orientation detection device.

For example, it is favourable for the second orientation detection device to comprise at least one inclination sensor and/or at least one compass sensor with which a change in orientation of the ultrasound probe may be detected.

Alternatively or additionally, it may advantageously be provided that the second orientation detection device be constructed spatially separately from the ultrasound probe, that the ultrasound probe comprise a marker device or such a marker device be arranged on the ultrasound probe and that preferably the second orientation detection device be configured for detecting radiation emitted and/or reflected by the marker device. It may in particular be provided in this embodiment that the first and the second orientation detection device be identical and be capable of detecting radiation emitted and/or reflected in each case by marker devices which are associated with the display device or to ultrasound probe.

It is favourable for the apparatus to comprise a tablet computer which comprises the display device. This makes it possible for the operator to position the display device in such a manner that he/she has a good view of the ultrasound image and is consequently able straightforwardly to associate structures of the ultrasound image with structures of the item under examination. By means of a change in orientation of the tablet computer in space, in particular rotation about an axis perpendicular to the ultrasound image plane, the ultrasound image on the display device is changed and in particular rotated in the opposite direction, so enabling a user-oriented display of the ultrasound image. In addition, as explained above, the ultrasound image may be displayed in device-oriented manner, a fixed spatial relationship preferably being provided between the ultrasound image and the ultrasound field. In particular, a change in orientation of the ultrasound probe in space may be detected, specifically rotation about an axis perpendicular to the ultrasound image plane and in this manner the image on the display may be changed and in particular rotated about an axis of rotation perpendicular to the ultrasound image plane.

The present invention is described below taking an ultrasound investigation of an item under examination in the form of a femur by way of example. It goes without saying, however, that the present invention may also be used for investigating any desired items and other bodies, for example for examining machine parts, vehicle parts etc. All that is essential is that the item under examination is capable of reflecting ultrasound radiation, such that, by point-by-point scanning of structures of the item under examination by means of ultrasound radiation, it is possible to determine the distance of the respective structure from an ultrasound probe and an ultrasound image may consequently be displayed.

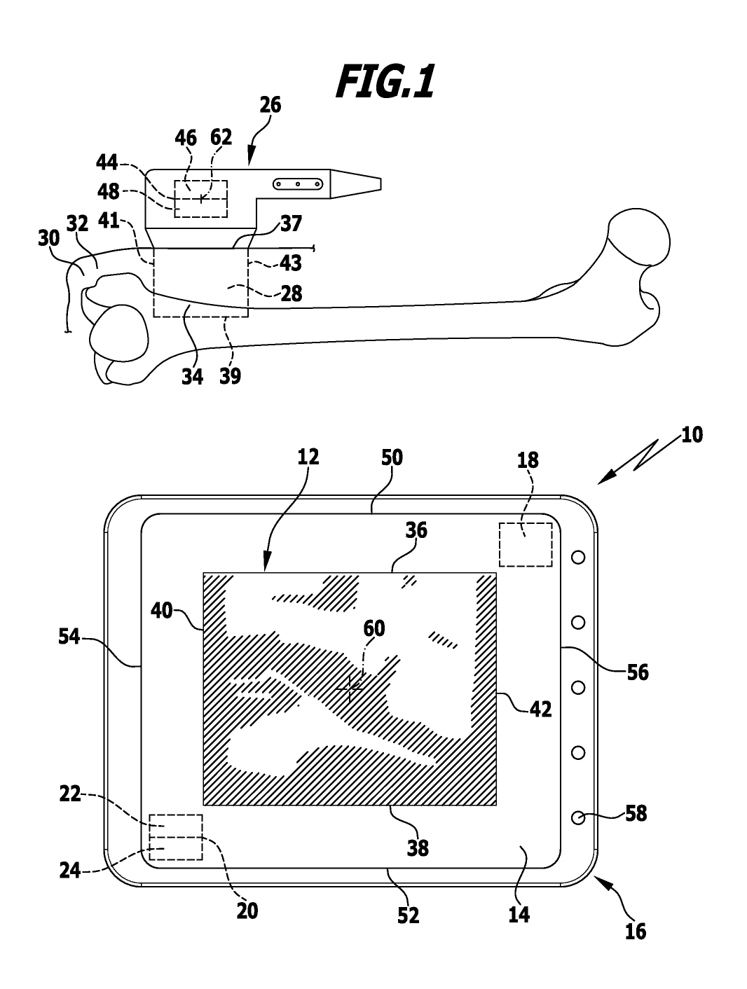

FIG. 1 shows a schematic view of a preferred embodiment of an apparatus according to an aspect of the invention, designated overall 10, for displaying an ultrasound image 12 (hereafter image 12) on a display device 14. The display device 14 is included in the case of the apparatus 10 by a tablet computer 16, which comprises a portable integrated electronic device which, in addition to the display device 14, also comprises an integrated data processing device 18. The data processing device 18 is only shown schematically in the drawings and comprises for example integrated electronic circuits or is constructed as an assembly of such integrated electronic circuits, for instance in the form of a microprocessor.

The data processing device 18 is configured and programmed so as to display the image 12 on the display device 14 by means of image signals which are supplied to the tablet computer 16 via for example wireless or wired data transfer interfaces which are not shown in the drawing. The data processing device 18 is furthermore configured and programmed so as to change, in particular to rotate and mirror, the display of the image 12 on the display device 14, this being explained in greater detail below. Where any mention is made below of a change to the image 12 on the tablet computer 16, in particular the display device 14 thereof, said change is initiated by the data processing device 18. Display of the image 12 on the tablet computer 16 means display of the image 12 on the integrated display device 14 thereof.

The tablet computer 16 furthermore comprises an orientation detection device 20, likewise shown only schematically, with which a change in orientation of the tablet computer 16 in space may be detected. The orientation detection device 20 comprises for this purpose a compass sensor 22 and an inclination sensor 24, such that a change in orientation of the tablet computer 16 in the event of rotation and/or panning about three spatial axes, in particular located perpendicularly to one another in pairs, may be detected. An associated change in orientation signal may be supplied to the data processing device 18 and evaluated by the latter.

It may furthermore be provided that the orientation detection device 20 also be configured to detect a change in location of the tablet computer 16 in space, for example in the event of movement thereof such that the tablet computer 16 is only moved in parallel to its original location in space, but not rotated.

If the orientation and location of the tablet computer 16 in space is changed in a manner which results from a possible parallel movement and rotation about more than just one spatial axis, the data processing device 18 may attribute this to superimposed movement. In particular, it is possible to identify rotation about an axis of rotation which is oriented perpendicularly to a plane defined by the image 12 (image plane), and rotational movements about axes of rotation extending in the image plane.

The apparatus 10 furthermore comprises an ultrasound probe 26 (hereafter probe 26), with which ultrasound radiation may be emitted in per se known manner and an ultrasound field 28 may be generated. The probe 26 comprises ultrasound transmitters arranged as a linear array, which are combined with corresponding ultrasound receivers, each of the ultrasound transmitters emitting ultrasound radiation and the ultrasound transmitters consequently together being capable of providing the ultrasound field 28.

The probe 26 may be used to examine an item under examination 30, in the present example a femur 32, the ultrasound field 28 penetrating in a manner known per se into the femur 32. The ultrasound transmitters and ultrasound receivers are combined with one another such that the radiation is in each case received at the location from which it has been emitted. The femur 32 is therefore examined point-by-point in the plane of the ultrasound field 28, the distance of a specific structure 34 from the probe 26 being obtained by the measured propagation time between emission and reception of the ultrasound radiation. In the transverse direction of the ultrasound field 28, the spatial arrangement of the individual regions of the structure 34 is obtained in that ultrasound radiation reflected from the respective regions of the structure 34 is received in different ultrasound receivers and it is consequently possible to resolve the structure 34 in the second spatial direction defined by the ultrasound field 28.

The composite image signal assembled from the contributions of the individual ultrasound receivers may be supplied to the tablet computer 16 and thus to the data processing device 18, for example by wired or wireless means, as shown in the drawings. The data processing device 18 processes the image signals and displays the image 12 on the display device 14, there being a fixed relationship between the image 12 and the geometry of the probe 26, apart from an exception which will be explained in greater detail below. For example, a top side 36 of the image 12 is associated with a top side 37 of the probe 26, the top side 37 being the region of the ultrasound field 28 located closest to the ultrasound transmitters and ultrasound receivers. A bottom side 38 of the image 12 is associated with a bottom side 39 of the probe 26 or the ultrasound field 28 thereof, which corresponds to that distance from the top side 37 which is still measured with the ultrasound field 28, i.e. by which radiation reflected by the structure 34 is still taken into account. A left-hand side 40 of the image 12 is associated with a left-hand side 41 of the probe 26, and a right-hand side 42 of the image 12 with a right-hand side 43 of the probe 26. The lengthwise direction of the sides 40 and 42 thus indicates the respective distance of a structure 34 from the probe 26, and the ultrasound radiation contributions received in each case by the ultrasound receivers are arranged side by side along the sides 36 and 38 transversely thereof.

The probe 26 likewise comprises an orientation detection device which is designated with the reference numeral 44. The orientation detection device 44 serves to detect a change in orientation of the probe 26 in space, in particular rotation about three spatial axes, in particular located perpendicularly to one another in pairs, and comprises for this purpose a compass sensor 46 and an inclination sensor 48. As has already been described above by way of example in relation to the orientation detection device 20, it may furthermore be provided that the orientation detection device 44 be capable of detecting a change in the location of the probe 26 in space, if said probe is moved in space with the ultrasound field 28 being moved in parallel in space.

A change in orientation and location of the probe 26 may be attributed to rotation thereof about an axis of rotation perpendicular to a plane defined by the ultrasound field 28 and to rotational movements of the probe 26 about axes of rotation which extend in the plane of the ultrasound field 28.

A change in orientation signal supplied by the orientation detection device 44 may be transferred to the data processing device 18 and taken into account thereby for displaying the image 12 on the display device 14.

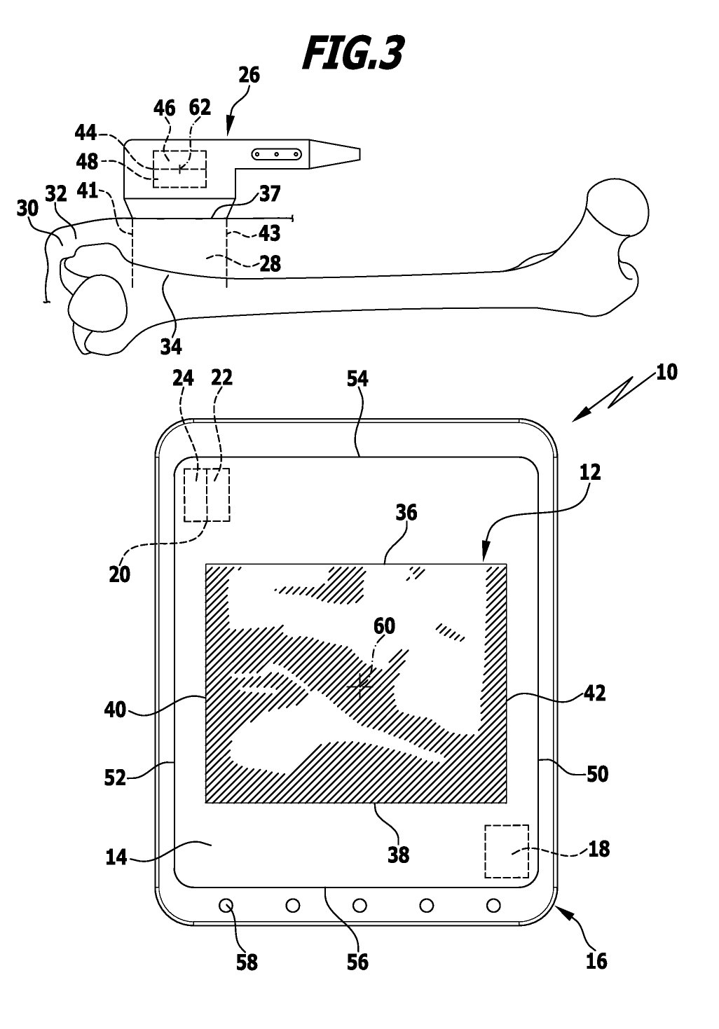

When using the apparatus 10, the operator may arrange the ultrasound probe 26 on the item under examination 30 such that the desired structure 34 is detected by the ultrasound field 28. Image signals are transferred to the tablet computer 16 and displayed by the data processing device 18 as an image 12 on the display device 14. In the same manner as with a conventional display of an ultrasound image, the image 12 is initially displayed such that the top side 36 is oriented parallel to an upper edge 50, the bottom side 38 parallel to a lower edge 52, the left-hand side 40 parallel to a left-hand edge 54 and the right-hand side 42 parallel to a right-hand edge 56 of the display device 14. The terms “top”, “bottom”, “left” and “right” for the edges 50 to 56 are here selected on the basis of the orientation of the tablet computer 16 shown in FIG. 1. In a different orientation of the tablet computer 16, for example the orientation shown in FIG. 3, the upper edge 50 is arranged on the right, the right-hand edge 56 at the bottom, the lower edge 52 on the left and the left-hand edge 54 at the top.

The operator may position the tablet computer 16 such that he/she has the best possible view of the image 12 and can associate the structures thereof with the structures 34 of the item under examination 30. In practice, however, it is sometimes desirable to position the tablet computer 16 in a different manner. At the same time, it is desirable for the operator still to have the best possible view of the image 12. This advantage may be achieved with the development described below of the tablet computer 16 and the apparatus 10.

The tablet computer 16 comprises an actuating element 58 the form of a push button or switch, actuation of which switches the tablet computer 16 over to a display mode in which the image 12 of the data processing device 18 is changed on the display device 14 as a function of a signal from the orientation detection device 20. This makes it possible to achieve a user-oriented display of the image 12 on the tablet computer 16, even if the orientation of the tablet computer 16 in space changes. In this manner, the operator still has a good view of the image 12 and is still able straightforwardly to associate the structures thereof with the structures 34 of the item under examination 30.

Once the actuating element 58 has been actuated, the data processing device 18 rotates the image 12 on the display device 14 if the latter is rotated in space about an axis of rotation 60 oriented perpendicularly to the image plane (FIGS. 1 to 3). As mentioned, rotation of the tablet computer 16 may be detected by means of the orientation detection device 20 and an associated change in orientation signal is transferred to the data processing device 18.

The data processing device 18 rotates the image 12 in the opposite direction to the rotation of the tablet computer 16 about the axis of rotation 60, and specifically by the same angular amount. This makes it possible to display the image 12 stationarily relative to the operator, in order to make it easier for him/her to associate structures of the image 12 with structures 34 of the item under examination 30.

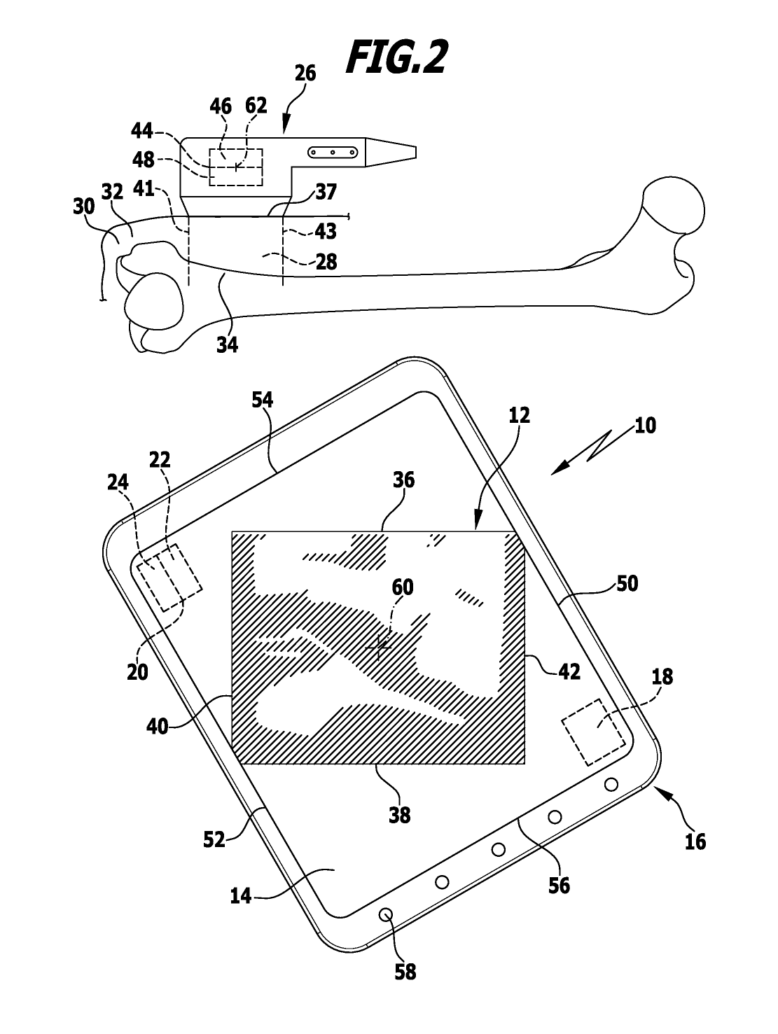

In comparison with the orientation of the tablet computer 16 shown in FIG. 1, the tablet computer is rotated in the orientation shown in FIG. 2 by roughly 30° about the axis of rotation 60, and, in the orientation shown in FIG. 3, by roughly 90° about the axis of rotation 60. Correspondingly, the data processing device 18 rotates the image 12 in FIG. 2 by roughly 30° in the opposite direction about the axis of rotation 60 and in FIG. 3 by roughly 90° in the opposite direction about the axis of rotation 60.

It may be provided that the data processing device 18 only rotates the image 12 if the angle of rotation of the tablet computer 16 exceeds a predeterminable threshold angle of rotation, but keeps the image 12 constant in the event of rotation of less than said angle of rotation. Slight movements of the tablet computer 16, for example due to its being knocked by the operator, are therefore disregarded in terms of rotation of the image 12. Flickering of the image 12 may be avoided in that, in this manner, it may be more recognised more readily by the operator.

In contrast, if the tablet computer 16 is rotated about an axis of rotation which extends in the image plane, for example parallel to the sides 36 and 38 or parallel to the sides 40 and 42, the data processing device 18 does not change the image 12 on the display device 14. If the tablet computer 16 is rotated or panned accordingly in the orientation shown in FIGS. 1 to 3 out of the plane of the drawing, the image 12 is not changed. In this manner, the image 12 is still readily recognisable for the operator.

Correspondingly, the image 12 is not changed if the tablet computer 16 is moved parallel in space.

The display mode of the tablet computer 16, in which the data processing device 18 changes the image 12 as a function of its change in orientation in space, may be switched off by actuating the actuating element 58 once again.

By making use of the orientation detection device 44 of the probe 26, it is furthermore possible to display the image 12 on the display device 14 in a manner in which the image 12 is oriented parallel to and thus in a fixed geometric relationship with the ultrasound field 28, relative to a projection of the image 12 in the plane defined by the ultrasound field 28. The image 12 may be displayed such that the top sides 36 and 37, the bottom sides 38 and 39, the left-hand sides 40 and 41 and the right-hand sides 42 and 43 are oriented parallel to one another. The data processing device 18 is capable of orienting the image 12 as a function of the signal supplied to it from the orientation detection device 44 of the probe 26. The geometry of the probe 26 is known to the data processing device 18. Using the signal from the orientation detection device 44, the data processing device 18 can therefore calculate how the ultrasound field 28 is oriented and display the image 12 accordingly on the display device 14. This additionally makes it easier for an operator to associate structures of the image 12 with structures 34 of the item under examination 30. Structures of the image 12 may, as it were, be shifted parallel into the item under examination 30 and located there.

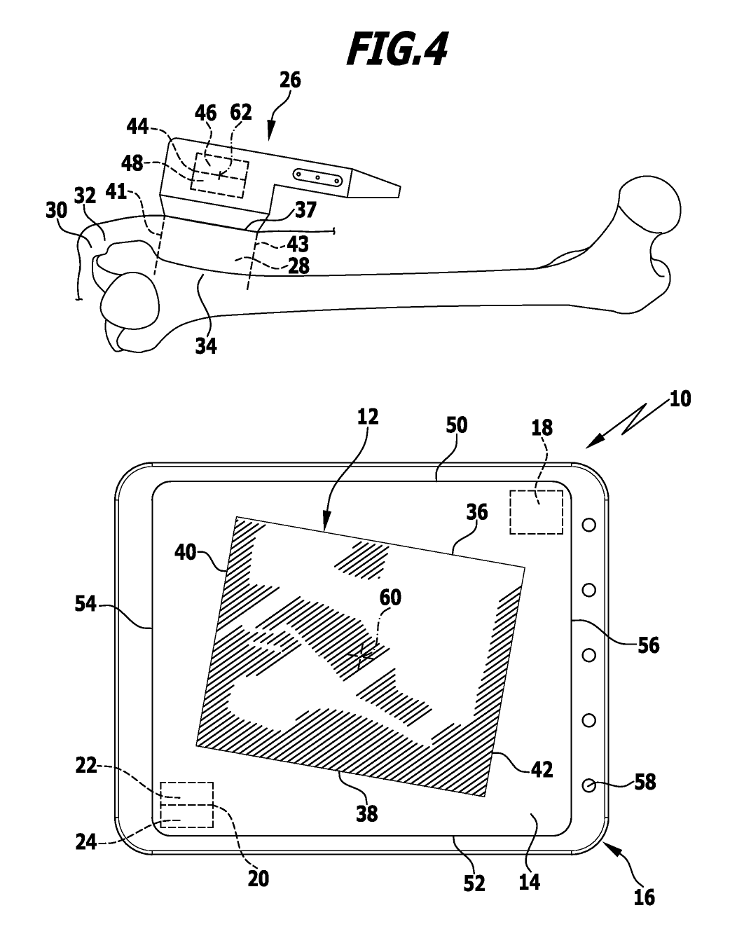

The above configuration of the apparatus 10 furthermore makes it possible to change the image 12 on the display device 14 as a function of a change in orientation of the probe 26 in space (FIGS. 1 and 4). If the probe 26 is rotated in space about an axis of rotation 62 which is oriented perpendicularly to the plane of the ultrasound field 28, the data processing device 18 rotates the image 12 on the display device 14 about an axis of rotation perpendicular to the image plane. In the present case, this axis of rotation coincides with the axis of rotation 60. The orientation detection device 44 may for this purpose supply a corresponding change in orientation signal to the data processing device 18, which evaluates said signal for rotating the image 12.

The image 12 is rotated in the same direction of rotation as the rotation of the probe 26 and by the same angular amount. It is furthermore also provided in this case that the image 12 is not rotated if the probe 26 is only rotated to such an extent that the angle of rotation is below a predeterminable threshold angle of rotation. In the event of only slight rotation of the probe 26, for example slight shaking by the operator, the image 12 is kept constant and is consequently more readily recognisable by the operator.

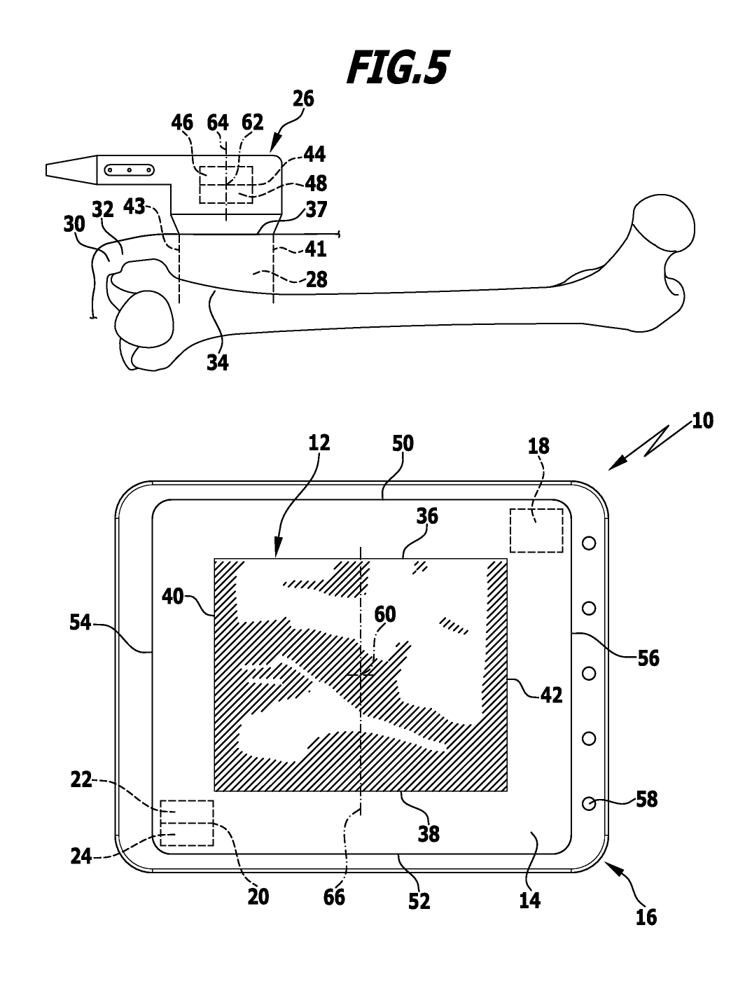

If, in contrast, the probe 26 is rotated about an axis of rotation which extends in the plane of the ultrasound field 28, the image 12 is not normally changed by the data processing device 18. There is, however, an exception to this (FIGS. 1 and 5). If the probe 26 is rotated about an axis of rotation 64 which extends parallel to the sides 41 and 43 through the centre of the ultrasound field 28, and by an angle of 180° or substantially 180°, the data processing device 18 mirrors the image 12. Mirroring occurs at a mirror plane 66 which is oriented perpendicular to the image plane and extends through the centre thereof parallel to the sides 40 and 42. This proves advantageous in order to make it easier for operator to recognise the image 12 and to associate the structures thereof with structures 34 of the item under examination 30.

When the image 12 is mirrored by the data processing device 18, unlike in the configuration shown in FIG. 1, the left-hand side 40 of the image 12 is associated with the right-hand side 43 of the ultrasound field 28. The right-hand side 42 is correspondingly associated with the left-hand side 41. In contrast, the top sides 36 and 37 and likewise the bottom sides 38 and 39 remain associated with one another. As a result, the image perceived by the operator on the display device 14 remains unchanged even once the probe 26 has been rotated about the axis of rotation 64. The operator is consequently saved the task of mirroring the image 12 in his/her head in order to establish the spatial relationship thereof relative to the ultrasound probe 26, thus to the ultrasound field 28, and to the examined structures 34.

Correspondingly, as has been explained above, in addition to two left/right orientation options, there are also two top/bottom orientation options in the apparatus 10. This functions in a corresponding manner to that explained above and is not shown separately in the drawings. If the probe 26 is rotated about an axis of rotation which extends in the plane of the ultrasound field 28 parallel to the sides 37 and 39 centrally through the ultrasound field 28, this results in mirroring of the image 12 by the data processing device 18 on the display device 14. Mirroring proceeds centrally through the image 12 about a mirror plane perpendicular to the mirror plane 66 and to the image plane. There are therefore also two top/bottom orientation options in the relative arrangement of probe 26 and the display device 14. This likewise makes it easier for an operator to recognise the image 12, since he/she does not have to turn the image 12 upside down mentally in order to associate the structures contained therein with the structures 34 of the item under examination 30.

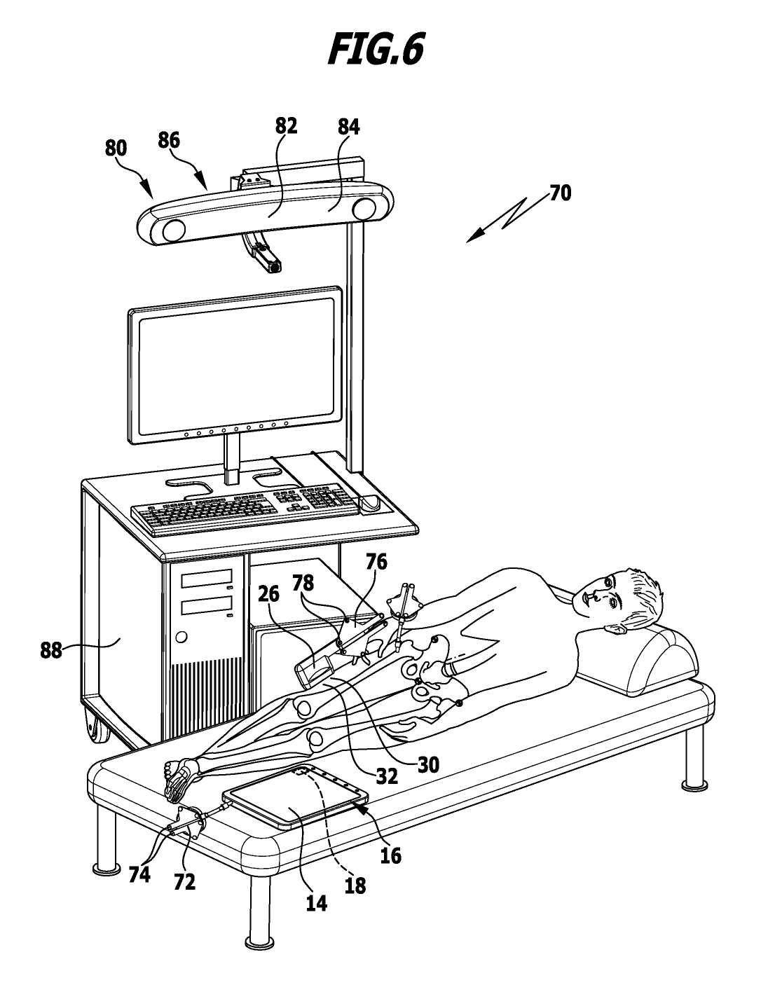

A further preferred embodiment of an apparatus according to an aspect of the invention is shown in FIG. 6, where it is designated overall with the reference numeral 70. The apparatus 70 comprises the tablet computer 16 and the probe 26, which are constructed as explained above, but do not necessarily comprise the orientation detection devices 20 and 44. A marker device 72 is fixed to the tablet computer 16, which marker device comprises marker elements 74 which are configured for reflecting electromagnetic radiation and in particular infrared radiation.

Similarly, a marker device 76 is fixed to the probe 26, which marker device comprises marker elements 78 which are capable of emitting electromagnetic radiation and in particular infrared radiation. Accordingly, the marker device 72 is a passive marker device and the marker device 76 an active marker device.

The apparatus 70 furthermore comprises a navigation system 80 with a detection apparatus 82 which for example takes the form of a stereo camera 84. The stereo camera 84 is capable of emitting electromagnetic radiation and in particular infrared radiation which is reflected by the marker elements 74. This radiation and the radiation emitted by the marker elements 78 may be detected by the stereo camera 84.

If the tablet computer 16 and/or the probe 26 are moved in space, the movement may be recognised by the stereo camera 84 in that there is a change in the direction from which it receives the radiation from the marker elements 74 and 78. This makes it possible to detect the location and orientation of the tablet computer 16 and the probe 26 in space and any changes thereto. In addition, the geometry of the marker devices 72 and 76 and their respective positional relationship to the tablet computer 16 and the probe 26 is likewise known.

The navigation system 80 is thus an orientation detection device 86 of the apparatus 70. A change in orientation signal of the navigation system 80 may be supplied to a data processing device 88 of the apparatus 70.

The above-described methods for changing the image 12 on the display device 14 may for example be carried out as follows with the apparatus 70:

On the one hand, image signals from the probe 26 may be transferred directly to the tablet computer 16. The data processing device 88 transfers the change in orientation signal to the data processing device 18 of the tablet computer 16, whereupon the data processing device 18 changes the image 12 on the display device as a function of a change in orientation of the tablet computer 16 and the probe 26 in space.

On the other hand, provision may be made for image signals from the probe 26 firstly to be transferred to the data processing device 88 and for the data processing device 88, taking account of the change in orientation signal of the navigation system 80, to process and rotate the image to be displayed by the display device 14 such that the image 12 appears in rotated manner on the display device 14. The change in orientation therefore takes place not in the data processing device 18, but instead in the data processing device 88.

Furthermore, the image 12 on the display device 14 may be changed with the apparatus 70 in a corresponding manner as has been explained above by way of example with apparatus 10 and with reference to FIGS. 1 to 5.

1. A method for displaying an ultrasound image on a display device, in which image signals are generated with an ultrasound probe during examination of an item under examination with ultrasound and transferred to a data processing device and the image signals are displayed by the data processing device as an ultrasound image on a display device, wherein, by means of an orientation detection device, a change in orientation of the display device in space is detected and an associated change in orientation signal is provided to the data processing device and the orientation of the ultrasound image on the display device is changed by the data processing device as a function of the change in orientation of the display device in space.

2. A method according to claim 1, wherein the ultrasound image is rotated on the display device in the opposite direction to the rotation of the display device, by the same angular amount, about an axis of rotation oriented perpendicularly to the ultrasound image plane.

3. A method according to claim 2, wherein the ultrasound image is not rotated if the angle of rotation of the display device is below a predeterminable threshold angle of rotation and is only rotated once the angle of rotation exceeds the threshold angle of rotation.

4. A method according to claim 1, wherein the orientation of the ultrasound image is not changed if the display device is rotated about an axis of rotation which extends in the ultrasound image plane.

5. A method according to claim 1, wherein the change in orientation of the ultrasound image proceeds as a function of a change in orientation of the display device subject to an actuating element being actuated by an operator, without which actuation the orientation of the ultrasound image is kept constant even in the event of a change in orientation of the display device in space.

6. A method according to claim 1, wherein an orientation detection device included in or arranged on the display device is used in order to detect the change in orientation of the display device in space, the display device and the data processing device being integrated in one another.

7. A method according to claim 1, wherein an orientation detection device spatially separate from the display device is used in order to detect the change in orientation of the display device in space.

8. A method according to claim 1, wherein the orientation of the ultrasound image on the display device is selected such that the ultrasound image is oriented parallel to the ultrasound field emitted by the ultrasound probe, relative to a projection of the ultrasound image on a plane defined by the ultrasound field.

9. A method according to claim 1, wherein, by means of an orientation detection device, a change in orientation of the ultrasound probe in space is detected and an associated change in orientation signal is provided to the data processing device and wherein the orientation of the ultrasound image on the display device is changed as a function of the change in orientation of the ultrasound probe in space.

10. A method according to claim 9, wherein, in the event of rotation of the ultrasound probe about an axis of rotation perpendicular to a plane defined by the ultrasound field, the ultrasound image on the display device is rotated about an axis of rotation oriented perpendicularly to the ultrasound image plane, in the same sense of rotation and by the same angular amount.

11. A method according to claim 9, wherein, in the event of rotation of the ultrasound probe by 180° or substantially 180° about an axis of rotation which extends in the plane of the ultrasound field and centrally therethrough, the ultrasound image on the display device is mirrored about a mirror plane perpendicular to the ultrasound image plane, which is oriented parallel to or contains the axis of rotation and extends through the centre of the ultrasound image.

12. A method according to claim 9, wherein an orientation detection device included in or arranged on the ultrasound probe is used in order to detect the change in orientation of the ultrasound probe in space.

13. A method according to claim 9, wherein an orientation detection device spatially separate from the ultrasound probe is used in order to detect the change in orientation of the ultrasound probe in space.

14. An apparatus for displaying an ultrasound image on a display device, the apparatus comprising an ultrasound probe for generating image signals during examination of an item under examination with ultrasound, a data processing device, to which the image signals are transferable and by which the image signals are processable, and a display device which is suppliable by the data processing device with ultrasound image information for displaying an ultrasound image, wherein the apparatus comprises a first orientation detection device for detecting a change in orientation of the display device in space and transferring an associated change in orientation signal to the data processing device and wherein the data processing device is configured and programmed such that it changes the orientation of the ultrasound image on the display device as a function of the change in orientation of the display device in space.

15. An apparatus according to claim 14, wherein the data processing device is configured and programmed such that it rotates the ultrasound image on the display device in the opposite direction to the rotation of the display device about an axis of rotation oriented perpendicularly to the ultrasound image plane, by the same angular amount.

16. An apparatus according to claim 15, wherein the data processing device is configured and programmed such that it does not rotate the ultrasound image if the angle of rotation of the display device is below a predeterminable threshold angle of rotation and only rotates it once the angle of rotation exceeds the threshold angle of rotation.

17. An apparatus according to claim 14, wherein the data processing device is configured and programmed such that it does not change the orientation of the ultrasound image on the display device if the display device is rotated about an axis of rotation which extends in the ultrasound image plane.

18. An apparatus according to claim 14, wherein the apparatus comprises an actuating element and the data processing device is configured and programmed such that it changes the orientation of the ultrasound image as a function of an actuation of the actuating element, without which actuation the data processing device keeps the orientation of the ultrasound image constant even in the event of a change in orientation of the display device in space.

19. An apparatus according to claim 14, wherein the display device comprises the first orientation detection device, the data processing device and the display device being integrated in one another.

20. An apparatus according to claim 19, wherein the first orientation detection device comprises at least of at least one of an inclination sensor and at least one compass sensor.

21. An apparatus according to claim 14, wherein the first orientation detection device is formed spatially separately from the display device, wherein the display device comprises a marker device or such a marker device is arranged on the display device and wherein the orientation detection device is configured for detecting radiation emitted and/or reflected by the marker device.

22. An apparatus according to claim 14, wherein the data processing device is configured and programmed such that it selects the orientation of the ultrasound image on the display device such that the ultrasound image is oriented parallel to the ultrasound field emitted by the ultrasound probe, relative to a projection of the ultrasound image on a plane defined by the ultrasound field.

23. An apparatus according to claim 14, wherein the apparatus comprises a second orientation detection device for detecting a change in orientation of the ultrasound probe in space and for transferring an associated change in orientation signal to the data processing device and wherein the data processing device is configured and programmed such that it changes the orientation of the ultrasound image on the display device as a function of the change in orientation of the ultrasound probe in space.

24. An apparatus according to claim 23, wherein the data processing device is configured and programmed such that, in the event of rotation of the ultrasound probe about an axis of rotation perpendicular to a plane defined by the ultrasound field, it rotates the ultrasound image on the display device about an axis of rotation oriented perpendicularly to the ultrasound image plane, in the same sense of rotation and by the same angular amount.

25. An apparatus according to claim 23, wherein the data processing device is configured and programmed such that, in the event of rotation of the ultrasound probe by 180° or substantially 180° about an axis of rotation which extends in the plane of the ultrasound field and centrally therethrough, it mirrors the ultrasound image on the display device about a mirror plane perpendicular to the ultrasound image plane, which is oriented parallel to or contains the axis of rotation and extends through the centre of the ultrasound image.

26. An apparatus according to claim 23, wherein the ultrasound probe comprises the second orientation detection device.

27. An apparatus according to claim 26, wherein the second orientation detection device comprises at least one of at least one inclination sensor and at least one compass sensor.

28. An apparatus according to claim 23, wherein the second orientation detection device is formed spatially separately from the ultrasound probe, wherein the ultrasound probe comprises a marker device or such a marker device is arranged on the ultrasound probe and wherein the second orientation detection device is configured for detecting radiation emitted and/or reflected by the marker device.

29. An apparatus according to claim 14, wherein the apparatus comprises a tablet computer including the display device.
